(19) United States
(12) Patent Application Publication
(10) Pub. No.: US 2021/0220301 A1
(43) Pub. Date: Jul. 22, 2021

(54) Pharmaceutical or Nutritional Combination Comprising Beta-Hydroxy-Betamethylbutyrate

(71) Applicant: Abbott Laboratories, Abbott Park, IL (US)

(72) Inventor: Shreeram SATHYAVAGEESWARAN, Singapore (SG)

(21) Appl. No.: 16/070,757

(22) PCT Filed: Jan. 16, 2017
(86) PCT No.: PCT/US2017/013672

§ 371 Date: Jul. 17, 2018

(30) Foreign Application Priority Data
Jan. 19, 2016 (GB) 1600990.4

(51) Int. Cl. A61K 31/19; A61K 31/12; A23L 33/00; A61K 9/00; A23L 2/38

(52) U.S. Cl. CPC A61K 31/19; A61K 31/12; A23L 2/38; A61K 9/0053; A61K 9/0095; A23L 33/30

(57) ABSTRACT

The present invention relates to a pharmaceutical or nutritional combination comprising beta-hydroxy beta-methylbutyrate (HMB) and tetrahydrocurcumin (THC) and methods comprising administering the combination to a subject. The methods of the invention relate to attenuating muscle protein degradation and/or preserving lean body mass in the subject.

### FIELD OF THE INVENTION

The present invention is directed to combinations comprising beta-hydroxy beta-methylbutyrate (HMB) and tetrahydrocurcumin (THC). Such combinations have been found to be particularly useful in attenuating muscle protein degradation, preserving lean body mass, or both in a subject in need thereof.

### BACKGROUND

Loss of lean body mass occurs with aging, muscle disuse, surgery, and a number of catabolic conditions including, but not limited to, cancer, burn injuries, chronic obstructive pulmonary disease, congestive heart failure, and chronic kidney disease. Such loss of lean body mass leads to functional decline, loss of independence, and increased disease burden. The loss of lean body mass is commonly attributed to an imbalance with the rate of muscle protein degradation outpacing the rate of muscle protein synthesis.

One compound that has been demonstrated to be useful in attenuating muscle protein degradation and preserving lean body mass is beta-hydroxy beta-methylbutyrate (HMB), a metabolite of branched chain amino acid, leucine. HMB has been shown to increase protein synthesis and decrease protein degradation. Traditionally, HMB has been used by athletes to enhance performance and build lean body mass. Recent research has focused on the use of HMB to preserve or rebuild muscle mass in populations with sarcopenia, immobility due to prolonged bed rest and post-surgery. Data have also demonstrated the benefits of HMB supplementation in preserving lean body mass and muscle strength in the elderly as well as in persons with chronic diseases such as AIDS and cancer (see for example WO 2012/092035 and WO 2012/097061). These benefits occur at a certain minimum dose of HMB (50 mg/kg/day for humans) (Deutz N E, Pereira S L, Hays N P, Oliver J S, Edens N K, Evans C M, et al. Effect of beta-hydroxy-beta-methylbutyrate (HMB) on lean body mass during 10 days of bed rest in older adults. Clin Nutr. 2013; 32:704-12). The beneficial effects of HMB have been postulated to be mediated by the Phosphatidylinositol 3-kinase (PI3K)/AKT serine/threonin protein kinase (AKT)/mammalian target of rapamycin kinase (mTOR) signaling pathway.

Nevertheless, HMB is expensive and compositions comprising HMB are difficult to prepare. This is because HMB is difficult to handle, requiring many special provisions that reduce manufacturing efficiency. There is therefore a need to develop compositions having a similar effect in attenuating muscle protein degradation and preserving lean body mass to existing HMB compositions.

### SUMMARY OF INVENTION

The present inventors have found that a combination comprising beta-hydroxy beta-methylbutyrate (HMB) and tetrahydrocurcumin (THC) can be used to attenuate muscle protein degradation, preserve lean body mass, or both in a subject in need thereof. Surprisingly, it has been found that if THC is used with HMB, a lower dose of HMB can be used to achieve attenuation of muscle protein degradation as compared with HMB-containing combinations without THC. This allows HMB to be administered at a lower dose than is normally expected to be effective (for example, half the conventional dose of 50 mg/kg/day).

In a first aspect, the present invention provides a pharmaceutical or nutritional combination comprising beta-hydroxy beta-methylbutyrate (HMB) and tetrahydrocurcumin (THC).

In a second aspect, the present invention provides a method of manufacturing a combination comprising beta-hydroxy beta-methylbutyrate (HMB) and tetrahydrocurcumin (THC), the method comprising mixing a source of HMB with a source of THC.

In a third aspect, the invention provides a method of treatment of a subject by therapy, the method comprising administering to the subject a pharmaceutical or nutritional combination comprising beta-hydroxy beta-methylbutyrate (HMB) and tetrahydrocurcumin (THC).

In a fourth aspect, the invention provides a method of attenuating muscle protein degradation and/or maintaining lean body mass in a subject in need thereof, the method comprising administering to the subject a pharmaceutical or nutritional combination comprising beta-hydroxy beta-methylbutyrate (HMB) and tetrahydrocurcumin (THC).

### BRIEF DESCRIPTION OF DRAWINGS

FIG. 1 shows the effects after 21 days of a composition comprising HMB and THC on soleus muscle mass (in grams) in rats that have been subjected to dexamethasone-induced muscle loss. Data for HMB alone and THC alone are also included as controls.

FIG. 2 shows the effects after 21 days of the same compositions on rectus femoris muscle mass (in grams) in the rats.

FIG. 3 shows the effects after 21 days of the same compositions on the lean mass of the rats (in grams).

FIG. 4 shows the effects after 21 days of the same compositions on the fore limb grip strength (g force) of the rats.

FIG. 5 shows the effects after 21 days of the same compositions on the hind limb grip strength (g force) of the rats.

### DETAILED DESCRIPTION

### A. Definitions

Unless defined otherwise, all technical and scientific terms used herein have the same meaning as commonly understood by the ordinary person skilled in the art to which the invention pertains. Without limiting any term, further clarifications of some of the terms used herein are provided below.

The term “pharmaceutical combination” as used herein refers to preparations which contain no additional components which are significantly toxic to the subjects to which the combination would be administered.

The phrase “pharmaceutically acceptable carrier” as used herein refers to a pharmaceutically acceptable material, combination or vehicle, suitable for administration to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to or impacting safety of the patient.

“Pharmaceutically acceptable excipients” (vehicles, additives) are those which can reasonably be administered to a subject mammal to provide an effective dose of the active ingredient employed. The term “excipient” refers to an agent which may be added to a formulation to provide a desired consistency, e.g., altering the bulk properties, to improve stability, and/or to adjust osmolality. Examples of commonly used excipients include, but are not limited to, sugars, polyols, amino acids, surfactants, and polymers.

The terms “nutritional formula” or “nutritional product” or “nutritional combination,” as used herein, are used interchangeably and, unless otherwise specified, refer to nutritional liquids, nutritional solids, nutritional semi-liquids, nutritional semi-solids, nutritional powders, nutritional supplements, and any other nutritional food product as known in the art. The nutritional powders may be reconstituted to form a nutritional liquid, all of which comprise one or more of fat, protein and carbohydrate, and are suitable for oral consumption by a human.

The term “nutritional liquid,” as used herein, unless otherwise specified, refers to nutritional products in ready-to-drink liquid form, concentrated form, and nutritional liquids made by reconstituting the nutritional powders described herein prior to use.

The term “nutritional powder,” as used herein, unless otherwise specified, refers to nutritional products in flowable or scoopable form that can be reconstituted with water or another aqueous liquid prior to consumption and includes both spray dried and dry-mixed/dry-blended powders.

The term “nutritional semi-solid,” as used herein, unless otherwise specified, refers to nutritional products that are intermediate in properties, such as rigidity, between solids and liquids. Some semi-solids examples include puddings, gelatins, and doughs.

The term “nutritional semi-liquid,” as used herein, unless otherwise specified, refers to nutritional products that are intermediate in properties, such as flow properties, between liquids and solids. Some semi-liquids examples include thick shakes and liquid gels.

The terms “retort” and “retort sterilized” are used interchangeably herein, and unless otherwise specified, refer to the common practice of filling a container, most typically a metal can or other similar package, with a nutritional liquid and then subjecting the liquid-filled package to the necessary heat sterilization step, to form a retort sterilized nutritional liquid product.

The terms “aseptic” and “aseptic sterilized” are used interchangeably herein, and unless otherwise specified, refer to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The terms “administer,” “administering,” “administered,” and “administration,” as used herein, unless otherwise specified, should be understood to include providing a nutritional combination to a subject, the act of consuming a nutritional combination (self-administration), and combinations thereof. In addition, it should be understood that the methods disclosed herein may be practised with or without doctor supervision or other medical direction.

The term “subject” as used herein, unless otherwise specified, refers to a mammal, including companion animals, livestock, laboratory animals, working animals, sport animals, and humans. In preferred embodiments, the subject is a human.

The term “subject in need thereof,” as used herein, unless otherwise specified, refers to a subject exhibiting increased muscle protein degradation, decreased muscle protein synthesis, loss of lean body mass, or combinations thereof. In certain exemplary embodiments, the increased muscle protein degradation, decreased muscle protein synthesis, or loss of lean body mass may be due to, at least in part, age, physical inactivity, injury, surgery, chronic disease, or combinations thereof. In certain exemplary embodiments, the subject in need thereof is an elderly human, optionally a physically inactive elderly human, optionally a diseased elderly human, and optionally both physically inactive and diseased. In certain exemplary embodiments, the subject in need thereof, is a human that is undergoing a temporary or permanent period of physical inactivity, due to disability, temporary injury, or healing from a surgery. In certain exemplary embodiments, the subject in need thereof has undergone hip surgery. In certain exemplary embodiments, the subject in need thereof has suffered a burn injury, for example, a third degree burn injury. In certain exemplary embodiments, the subject in need thereof is a human undergoing rehabilitation (i.e., physical rehabilitation) due to disease, injury, surgery, hospital admission, and combinations thereof. In certain exemplary embodiments, the subject in need thereof is a human with a chronic disease condition such as, for example, cancer, cachexia, COPD, or end stage renal disease. In certain exemplary embodiments, the subject in need thereof is a human being treated with glucocorticoids for an extended period of time. In certain exemplary embodiments, the subject in need thereof is a human suffering from a muscle disease such as, for example, muscular dystrophy.

The term “elderly,” as used herein, unless otherwise specified, refers to a human of at least 45 years of age, including at least 50 years of age, at least 55 years of age, at least 60 years of age, at least 65 years of age, at least 70 years of age, at least 75 years of age, and including at least 80 years of age or greater. The term “elderly” also includes humans of 45 years of age to 100 years of age, and humans of 55 years of age to 80 years of age.

The term “lean body mass,” as used herein, unless otherwise specified, refers to the amount or size of muscle or muscle groups, as expressed by muscle weight, mass, area, or volume. Muscle mass may also be expressed as total muscle mass, lean body mass of a body compartment such as the leg, or cross-sectional area of a leg or arm compartment.

The volume or mass of the muscle can be determined using any known or otherwise effective technique that provides muscle area, volume, or mass such as DEXA, or using visual or imaging techniques such as MRI or CT scans.

The term “muscle,” as used herein, unless otherwise specified, refers to skeletal muscle and other non-skeletal, striated muscles such as diaphragm, extraocular muscle, and so forth.

The term “effective amount,” as used herein, unless otherwise specified, refers to a sufficient amount of a combination to exhibit a therapeutic effect (e.g., attenuate muscle protein degradation, preserve lean body mass). The exact amount required to achieve the desired effect will vary from subject to subject, depending on the species, age, weight, lifestyle and general condition of the particular subject.

The term “serving” as used herein, unless otherwise specified, refers to an amount of the combination which is intended to be consumed by an individual in one sitting or within one hour or less. In certain exemplary embodiments, the combination is packaged as a single serving. In certain exemplary embodiments, the combination is packaged in a container containing multiple servings, wherein the container bears instructions on how to separate a single serving of the combination from the bulk combination. Unless otherwise specified, when referring to a liquid combination, the term serving refers to a 230 mL portion. Unless otherwise specified, when referring to a solid combination, the term serving refers to a 54 g serving.

The term “THC”, as used herein, unless otherwise specified, refers to tetrahydrocurcumin, and other suitable sources of THC such as the free acid, salts (both organic and inorganic), anhydrous salts, esters (including the methyl ester, ethyl ester, phosphoesters, etc.), lactones and other bioavailable forms of HMB suitable for oral administration. THC is also known as white curcumin and 1,7-bis(4-hydroxy-3-methoxyphenyl)heptane-3,5-dione.

The term “HMB,” as used herein, unless otherwise specified, refers to β-hydroxy-β-methylbutyrate (or beta-hydroxy-beta-methylbutyrate), and other suitable sources of HMB such as the free acid, salts (both organic and inorganic), anhydrous salts, esters (including the methyl ester, ethyl ester, phosphoesters, etc.), lactones and other bioavailable forms of HMB suitable for oral administration.

All percentages, parts and ratios as used herein, are by dry weight of the total combination, unless otherwise specified. All such weights, as they pertain to listed ingredients, are based on the active level and, therefore, do not include solvents or by-products that may be included in commercially available materials, unless otherwise specified. For example, where the combination is a solid, the percentages are based on the total weight of the combination prior to reconstitution. Where the combination is a liquid, the percentages are based on the total weight of the combination minus the solvent, in other words by dry weight of the combination.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

All combinations of method or process steps as used herein can be performed in any order, unless otherwise specified or clearly implied to the contrary by the context in which the referenced combination is made.

In the following passages different aspects of the invention are defined in more detail. Each aspect so defined may be combined with any other aspect or aspects unless clearly indicated to the contrary.

### B. Combinations of the Invention and Methods of Manufacture

In a first aspect, the present invention is directed to a pharmaceutical or nutritional combination comprising, consisting essentially of or consisting of beta-hydroxy beta-methylbutyrate (HMB) and tetrahydrocurcumin (THC). In certain preferred embodiments, the combination is a nutritional combination.

At a certain minimum dose (typically 50 mg/kg/day for humans), HMB is known to be useful in preserving or rebuilding muscle mass and/or strength, for example in populations with sarcopenia, the elderly and persons with chronic diseases like AIDS and cancer. Surprisingly, the present inventors have found that the inclusion of THC enables the minimum effective dose of HMB to be reduced. This is advantageous because HMB is expensive to incorporate into nutritional combinations, partly due to ingredient costs but also because it is difficult to handle, requiring many special provisions that reduce manufacturing efficiency. Accordingly, the present invention allows combinations to be prepared having lower levels of HMB without compromising the effect on preserving or rebuilding muscle mass and/or strength.

HMB is a metabolite of the essential amino acid leucine, and has the IUPAC name 3-hydroxy-3-methylbutanoic acid. One suitable form of HMB that may be utilized in the exemplary nutritional combinations and methods described herein is the calcium salt of HMB, also designated as Ca-HMB, which is most typically the monohydrate calcium salt. The HMB used in the combinations of the exemplary embodiments may be obtained from any suitable source. Calcium HMB monohydrate (Ca-HMB) is commercially available from Technical Sourcing International (TSI) of Salt Lake City, Utah. The amounts of HMB to be incorporated into the combinations described herein are based on the calcium salt form of HMB. It would be within the capabilities of the skilled person to adjust the quantity depending on the source used to provide the desired amount of the free acid.

Although the monohydrate of the calcium salt may be utilized in certain exemplary embodiments of the nutritional combinations and methods disclosed herein, other suitable sources or forms include HMB as a free acid, a salt, an anhydrous salt, an ester, a lactone, or other product forms that provide a bioavailable form of HMB suitable for administration. Non-limiting examples of suitable salts of HMB include HMB salts, hydrated or anhydrous, of sodium, potassium, chromium, calcium, or other non-toxic salt forms.

In certain exemplary embodiments, the combination comprises from 0.5 to 2 wt % HMB by dry weight of the combination, preferably from 1 to 2 wt %, still more preferably from 1.3 to 1.7 wt %, and most preferably about 1.5 wt %. Thus, the exemplary combinations comprise significantly less HMB than conventional HMB-containing nutritional combinations used to preserve lean body mass or attenuate muscle protein degradation, which typically comprise about 3 wt % HMB by dry weight of the combination.

In certain exemplary embodiments, the combination comprises from 0.6 to 1.2 grams of HMB per serving, preferably from 0.7 to 0.9 grams per serving, and most preferably about 0.75 grams per serving. The serving size is as defined above. Thus, the exemplary combinations comprise significantly less HMB than conventional HMB-containing nutritional combinations, which typically comprise about 1.5 grams per serving HMB where a serving is defined on the above basis.

THC has been shown to exhibit protective physiological and pharmacological properties similar to curcumin (also known as yellow curcumin, found in ginger), such as anti-oxidant, radical scavenging, anti-metastatic, and anti-carcinogenic activities. However, while HMB has been widely reported to prevent muscle loss, there are no reports on the efficacy of THC against muscle loss. As is demonstrated in the Examples, the present inventors have surprisingly found that a combination of HMB and THC has a greater effect than the sum of the components individually. Thus, there is a synergy between the two components that allows the amount of HMB included in the formulation to be reduced.

The amounts of THC to be incorporated into the combinations described herein are based on the free acid. It would be within the capabilities of the skilled person to adjust the quantity depending if a salt form was used to provide the desired amount of the free acid.

In certain exemplary embodiments, the combination comprises from 0.5 to 1.5 wt % THC by dry weight of the composition, more preferably from 0.6 to 1.2 wt % and most preferably from 0.8 to 1 wt %.

In certain exemplary embodiments, the combination comprises from 0.05 to 3 grams of THC per serving, preferably from 0.2 to 1 grams per serving, more preferably from 0.2 to 0.8 grams per serving, and most preferably from 0.4 to 0.6 grams per serving. The serving size is as defined above.

In certain exemplary embodiments, the combination has a weight ratio of HMB to THC of from 1:1 to 5:1, preferably from 1:1 to 3:1, and most preferably from 1:1 to 2:1.

In certain exemplary embodiments, the combination is a nutritional combination and comprises from 1 to 2 wt % HMB and from 0.5 to 1.5 wt % THC by dry weight of the combination.

In certain exemplary embodiments, the combination is a nutritional combination and comprises from 0.6 to 1.2 grams per serving HMB and from 0.2 to 0.8 grams per serving THC, wherein a serving as defined as above.

In certain exemplary embodiments, the HMB and THC are provided in separate compositions.

In certain preferred embodiments, the HMB and THC are provided in a single composition. Preferably, the combination consists of a single composition comprising HMB and THC. In other words, the combination is preferably a composition comprising HMB and THC.

### Additional Ingredients

In certain exemplary embodiments, the combination is a pharmaceutical combination. In other words, the combination contains no additional components which are significantly toxic to the subjects to which the combination would be administered. Preferably, where the combination is a pharmaceutical combination, the combination comprises one or more pharmaceutically acceptable excipients. As noted above, examples of commonly used excipients include, but are not limited to, sugars, polyols, amino acids, surfactants, and polymers.

In certain preferred embodiments, the combination is a nutritional combination. In other words, the combination comprises at least one of a protein, a carbohydrate or a fat.

In certain exemplary embodiments, the nutritional combination comprises a source of protein. In one exemplary embodiment, the source of protein is present in an amount sufficient to provide about 3 to about 30 grams, about 5 to about 15 grams, or about 7 to about 10 grams of protein per serving. Alternatively, the amount of protein present in the combination may be expressed in terms of percentage of protein by dry weight of the combination (wt %). In such instances, the combinations may comprise a source of protein in an amount sufficient to provide 5 to 50 wt % protein by dry weight of the combination, preferably from 10 to 30 wt %, more preferably from 10 to 20 wt %, and most preferably from 12 to 18 wt %.

Various proteins, including one protein or more than one protein, may be utilized to provide the source of protein in nutritional combinations according to the exemplary embodiments. Proteins suitable for use in the nutritional combinations according to the embodiments disclosed herein include, but are not limited to, hydrolyzed, partially hydrolyzed or non-hydrolyzed proteins or protein sources, and can be derived from any known or otherwise suitable source such as milk (e.g., casein, whey), animal (e.g., meat, fish), cereal (e.g., rice, corn), vegetable (e.g., soy, pea, potato), or combinations thereof.

Non-limiting examples of the source of protein include whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, skim milk, low fat milk, nonfat dry milk, skim milk powder, condensed skim milk, soy protein concentrates, soy protein isolates, soy protein hydrolysates, pea protein concentrates, pea protein isolates, pea protein hydrolysates, collagen proteins, collagen protein isolates, insect proteins, earthworm proteins, potato protein, rice protein, corn protein, wheat protein, sunflower protein, chickpea protein, quinoa protein, and combinations thereof.

In certain exemplary embodiments, the nutritional combination comprises a source of carbohydrates. In certain exemplary embodiments, the source of carbohydrates is present in an amount sufficient to provide about 10 to about 100 grams of carbohydrates per serving. The source of carbohydrates may be from one carbohydrate or more than one carbohydrate. In certain exemplary embodiments, the nutritional combinations comprise a source of carbohydrates in an amount sufficient to provide about 10 to about 40 grams, about 15 to about 35 grams, about 25 to about 35 grams, or about 28 to about 32 grams of carbohydrates per serving. Alternatively, the amount of carbohydrates present in the combination may be expressed in terms of percentage of carbohydrates by dry weight of the combination (wt %). In such instances, the combinations may comprise a source of carbohydrates in an amount sufficient to provide from 20 to 70 wt % carbohydrates by dry weight of the combination, preferably from 30 to 60 wt %, more preferably from 40 to 60 wt %, and most preferably from 50 to 60 wt %.

Carbohydrates used in the embodiments disclosed herein may be simple, complex, or variations or combinations thereof. Generally, any source of carbohydrates may be used so long as it is suitable for use in oral nutritional combinations and is otherwise compatible with any other selected ingredient or feature present in the nutritional combination. Non-limiting examples of a source of carbohydrates which may be suitable for use in the exemplary nutritional combinations described herein include maltodextrin, hydrolyzed or modified starch or cornstarch, glucose polymers, corn syrup, corn syrup solids, rice-derived carbohydrates, sucrose, glucose, fructose, lactose, high fructose corn syrup, honey, sugar alcohols (e.g., maltitol, erythritol, sorbitol), isomaltulose, sucromalt, pullulan, potato starch, and other slowly-digested carbohydrates, dietary fibers including, but not limited to, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low and high methoxy pectin, cereal beta-glucans (e.g., oat beta-glucan, barley beta-glucan), carrageenan and psyllium, Fibersol™, other resistant starches, and combinations thereof.

In certain exemplary embodiments, the nutritional combination comprises a source of fructooligosaccharides (FOS). The FOS, in certain exemplary embodiments, may be natural or synthetic, short-chain or long-chain, and combinations thereof. Natural sources of FOS include, but are not limited to, Jerusalem artichoke, chicory root, yacón, bananas, onions, garlic, asparagus, barley, wheat, jicama, and leeks. Suitable synthetic sources of FOS include Actilight® (Beghin-Meiji, Marckolsheim, France) and scFOS® (Ingredion, Inc., Westchester, Ill.). In certain exemplary embodiments, the nutritional combinations comprise a source of FOS in an amount sufficient to provide about 50 mg to about 1.5 grams, about 100 mg to about 500 mg, or about 150 mg to about 300 mg of FOS per serving. Alternatively, the amount of FOS present in the combination may be expressed in terms of percentage of FOS by dry weight of the combination (wt %). In such instances, the combinations may comprise FOS in an amount sufficient to provide from 0.1 to 2 wt % FOS by dry weight of the composition, preferably from 0.2 to 1 wt %, and most preferably from 0.3 to 0.6 wt %.

In certain exemplary embodiments, the nutritional combination comprises a source of fat. In certain exemplary embodiments, the source of fat is present in an amount sufficient to provide about 5 to about 20 grams or about 5 to about 15 grams of fat per serving. Alternatively, the amount of fat present in the combination may be expressed in terms of percentage of fat by dry weight of the combination (wt %). In such instances, the combinations may comprise fat in an amount sufficient to provide from 5 to 50 wt % fat by dry weight of the combination, preferably from 10 to 30 wt %, more preferably from 10 to 20 wt %, and most preferably from 12 to 18 wt %.

Non-limiting examples of fats suitable for use in the exemplary nutritional combinations include canola oil, corn oil, coconut oil, fractionated coconut oil, soy oil, olive oil, safflower oil, high GLA safflower oil, high oleic safflower oil, MCT oil (medium chain triglycerides), sunflower oil, high oleic sunflower oil, palm and palm kernel oils, palm olein, marine oils, cottonseed oils, algal and fungal derived oils, and combinations thereof.

In certain preferred embodiments, the combination is a nutritional composition comprising:
from 1 to 2 wt % HMB;
from 0.5 to 1.5 wt % THC;
a source of protein in an amount sufficient to provide from 10 to 30 wt % protein;
a source of carbohydrates in an amount sufficient to provide from 30 to 60 wt % carbohydrates; and
a source of fat in an amount sufficient to provide from 10 to 30 wt % fat;
all by dry weight of the composition.

In certain preferred embodiments, the combination is a nutritional composition comprising:
from 1 to 2 wt % HMB;
from 0.5 to 1.5 wt % THC;
a source of protein in an amount sufficient to provide from 10 to 30 wt % protein;
a source of carbohydrates in an amount sufficient to provide from 30 to 60 wt % carbohydrates; and
a source of fat in an amount sufficient to provide from 10 to 30 wt % fat;
all by dry weight of the composition.

In certain preferred embodiments, the combination is a nutritional composition comprising: from 1.3 to 1.7 wt % HMB;
from 0.6 to 1.2 wt % THC;
a source of protein in an amount sufficient to provide from 10 to 30 wt % protein;
a source of carbohydrates in an amount sufficient to provide from 30 to 60 wt % carbohydrates; and
a source of fat in an amount sufficient to provide from 10 to 30 wt % fat;
all by dry weight of the composition.

In certain preferred embodiments, the combination is a nutritional composition comprising:
from 1.3 to 1.7 wt % HMB;
from 0.6 to 1.2 wt % THC;
a source of protein in an amount sufficient to provide from 12 to 18 wt % protein;
a source of carbohydrates in an amount sufficient to provide from 40 to 60 wt % carbohydrates; and
a source of fat in an amount sufficient to provide from 12 to 18 wt % fat;
all by dry weight of the composition.

In certain exemplary embodiments, the nutritional combination comprises at least one vitamin and at least one mineral. For example, in certain exemplary embodiments, the nutritional combination comprises vitamins and minerals that have antioxidant properties such as vitamin E, Vitamin C, selenium, molybdenum, and combinations thereof. In certain exemplary embodiments, the nutritional combination comprises vitamin D, including vitamin D₂ and vitamin D₃, which promotes intestinal absorption of calcium and phosphate. In certain exemplary embodiments, the nutritional combination comprises about 125 to about 200 IUs or about 150 to about 175 IUs of vitamin D (1 IU of vitamin D is equivalent to 0.025 micrograms of vitamin D) per serving.

In certain exemplary embodiments, the nutritional combinations may include other vitamins and related nutrients, non-limiting examples of which include vitamin A, vitamin A palmitate, vitamin E acetate, vitamin C palmitate (ascorbyl palmitate), vitamin K, thiamine, riboflavin, pyridoxine, vitamin B₁₂, carotenoids (e.g., beta-carotene, zeaxanthin, lutein, lycopene), niacin, folic acid, pantothenic acid, biotin, choline, inositol, salts and derivatives thereof, and combinations thereof. In certain exemplary embodiments, the nutritional combinations comprise any of a variety of additional minerals, non-limiting examples of which include calcium, potassium, iodine, phosphorus, magnesium, iron, zinc, manganese, copper, sodium, chromium, chloride, and combinations thereof.

The exemplary nutritional combinations can provide up to about 500 kcal of energy per serving, including from 240 to 500 kcal, from 275 to 450 kcal, from 300 to 400 kcal, from 325 to 375 kcal, or from 325 to 350 kcal per serving.

In certain exemplary embodiments, the nutritional combination comprises one or more functional ingredients that promote muscle health. For example, in certain exemplary embodiments, the one or more functional ingredients are selected from at least one of: a branched-chain amino acid selected from the group consisting of leucine, isoleucine, valine, metabolites of any of the foregoing branched-chain amino acids, and combinations thereof; α-ketoisocaproic acid (KIC); α-hydroxyisocaproic acid (HICA); β-alanine; a green tea catechin selected from the group consisting of epigallocatechin gallate, epigallocatechin, epicatechin, catechin, epicatechin gallate, and metabolites thereof; creatine; carnitine; carnosine; taurine; anserine; one or more of the proteinogenic amino acids; curcumin; green tea extract; plum extract; resveratrol; α-tocopherol; and combinations thereof.

In certain exemplary embodiments, the nutritional combination may comprise other optional ingredients that may modify the physical, chemical, aesthetic or processing characteristics of the nutritional combination, or serve as additional nutritional components. Many such optional ingredients are known or otherwise suitable for use in medical food or other nutritional products and may also be used in the nutritional combinations described herein, provided that such optional ingredients are safe for oral administration and are compatible with the essential and other ingredients in the selected product form.

In certain exemplary embodiments, the combination may comprise at least one sweetening agent. In certain exemplary embodiments, the at least one sweetening agent is a sugar alcohol such as maltitol, erythritol, sorbitol, xylitol, mannitol, isomalt, and lactitol, or at least one artificial or high potency sweetener such as acesulfame K, aspartame, sucralose, saccharin, stevia, and tagatose, and combinations thereof. The sweetening agents, especially as a combination of a sugar alcohol and an artificial sweetener, can be useful in formulating liquid nutritional combinations having a desirable favor profile. These sweetener combinations can also be effective in masking undesirable flavors, for example, as sometimes associated with the addition of vegetable proteins to a liquid nutritional combination.

In certain exemplary embodiments, the combination may comprise a flowing agent or anti-caking agent to retard clumping or caking of a powder embodiment over time and to make the powder flow easily from its container. Any flowing or anti-caking agents that are known or otherwise suitable for use in a powder are suitable for use herein, non-limiting examples of which include tricalcium phosphate, silicates, and combinations thereof. The concentration of the flowing agent or anti-caking agent will often vary depending upon the product form, the other selected ingredients, the desired flow properties, and so forth.

In certain exemplary embodiments, the combination may comprise a stabilizer. Any stabilizer that is known or otherwise suitable for use in a nutritional combination may also be suitable for use herein, non-limiting examples of which include gums such as xanthan gum and locust bean gum.

In certain exemplary embodiments, the combination optionally includes one or more masking agents to reduce or otherwise obscure the development of any residual bitter flavors and after taste in the combination over time. Suitable masking agents include natural and artificial sweeteners, sodium sources such as sodium chloride, and hydrocolloids such as guar gum, xanthan gum, carrageenan, gellan gum, and combinations thereof. The amount of masking agent used will often vary depending upon the particular masking agent selected, other ingredients in the formulation, and other formulation or product target variables.

Exemplary embodiments of the combinations may also be substantially free of any optional or selected essential ingredient or feature described herein, provided that the remaining product still contains some of the required ingredients or features as described herein. In this context, and unless otherwise specified, the term “substantially free” means that the selected nutritional product contains less than a functional amount of the noted optional or selected essential ingredient, typically less than 1.0%, including less than 0.5%, less than 0.1%, and zero percent, by weight of such optional or selected essential ingredient.

In certain exemplary embodiments, the combination is formulated in a product form suitable for oral administration. Oral administration, as defined herein, includes any form of administration in which the combination is introduced into the subject's digestive system, including the stomach and small intestine. For example, oral administration includes nasogastric intubation, in which a tube is run from through the nose to the stomach of the subject to administer food or drugs. Suitable forms of such combinations may include liquids, powders, solids, semi-solids, semi-liquids combinations, provided that such a formulation allows for the effective delivery and consumption of the nutritional combination.

In certain exemplary embodiments, the combination may be a solid, liquid, semi-solid, semi-liquid, or powder. Where the combination is a nutritional combination, examples of combination forms suitable for use herein include snack and meal replacement products, including those formulated as bars; sticks; cookies; breads, cakes, or other baked goods; frozen liquids; candy; breakfast cereals; powders, granulated solids, or other particulates; snack chips or bites; frozen or retorted entrees; and so forth. In certain exemplary embodiments, the nutritional combination can be in a form that falls between solid and liquid, such as puddings, yogurts, or gels. In certain exemplary embodiments, when the nutritional combination is a solid product, a serving thereof may be about 70 grams.

Examples of suitable liquid nutritional combinations include snack and meal replacement products, hot or cold beverages, carbonated or non-carbonated beverages, juices or other acidified beverages, milk or soy-based beverages, shakes, coffees, teas, and so forth. These liquid combinations are most typically formulated as suspensions or emulsions, but can also be formulated in any other suitable form such as clear liquids, substantially clear liquids, liquid gels, and so forth. In certain exemplary embodiments, when the nutritional combination is a liquid nutritional product, a serving thereof may be about 115 to 237 milliliters (˜8 fl. oz.).

In a second aspect, the present invention is directed to a method of manufacturing a combination as defined herein elsewhere. The method comprises mixing a source of THC with a source of HMB. Useful sources of THC and HMB are as described above. All embodiments of the combinations described herein in connection with the first aspect of the invention are equally applicable to the method of manufacture in accordance with the second aspect of the invention.

The exemplary nutritional liquids may be prepared by any suitable process or method. In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing selected oils (e.g., canola oil, corn oil) and then adding an emulsifier (e.g., soy lecithin), fat soluble vitamins, and a portion of the total protein (e.g., milk protein concentrate) with continued heat and agitation. The CHO-MIN slurry is formed by adding with heated agitation to water: minerals (e.g., potassium citrate, magnesium phosphate, calcium carbonate), trace minerals and ultra trace minerals (e.g., TM/UTM premix), thickening or suspending agents (e.g., gellan gum, carrageenan), HMB and THC. The resulting CHO-MIN slurry is held for 10 minutes with continued heat and agitation before adding additional minerals (e.g., potassium chloride, magnesium carbonate, potassium iodide), and carbohydrates (e.g., fructooligosaccharide, sucrose). The PIW slurry is then formed by mixing with heat and agitation the remaining protein (e.g., sodium caseinate, soy protein isolate, whey protein concentrate) into water.

The resulting slurries are then blended together with heated agitation and the pH adjusted to a desired range, typically from 6.6-7.0, after which the combination is subjected to high-temperature short-time (HTST) processing during which the combination is heat treated, emulsified and homogenized, and then allowed to cool. Water soluble vitamins and ascorbic acid are added, the pH is again adjusted to the desired range (if necessary), flavors are added, and water is added to achieve a desired total solid level. The combination is then aseptically packaged to form an aseptically packaged nutritional emulsion, or the combination is added to retort stable containers and then subjected to retort sterilization to form retort sterilized nutritional emulsions.

The manufacturing processes for the nutritional emulsions may be carried out in ways other than those set forth herein without departing from the spirit and scope of the present general inventive concepts. The present embodiments are, therefore, to be considered in all respects illustrative and not restrictive with changes and equivalents intended to fall within the general inventive concepts.

A nutritional powder, such as a spray dried nutritional powder, may be prepared by any combination of known or otherwise effective techniques suitable for making and formulating a spray dried nutritional powder. The spray drying step may likewise include any spray drying technique that is known for or otherwise suitable for use in the production of nutritional powders. Many different spray drying methods and techniques are known for use in the nutrition field, of which many are suitable for use in the manufacture of the spray dried nutritional powders herein.

One method of preparing an exemplary spray dried nutritional powder comprises forming and homogenizing an aqueous slurry or liquid comprising HMB, THC, protein, carbohydrates, and fat, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder. In certain exemplary embodiments, the methods of manufacture utilize calcium HMB. As previously discussed, the calcium HMB is most typically formulated as a monohydrate salt.

Nutritional combinations according to the exemplary embodiments are useful for providing sole, primary, or supplemental sources of nutrition, as well as providing one or more of the benefits as described herein such as attenuating muscle protein degradation, preserving lean body mass, or both.

### C. Methods of the Invention

In a third aspect, the present invention is directed to a method of treatment of a subject by therapy, the method comprising administering to the subject the combination described elsewhere herein. The present invention also encompasses a combination for use in a method of treatment of a subject by therapy wherein the combination is as described herein elsewhere.

In a fourth aspect, the present invention is directed to a method of attenuating muscle protein degradation and/or maintaining lean body mass in a subject in need thereof, the method comprising administering to the subject the combination described elsewhere herein. The present invention also encompasses a combination for use in a method of attenuating muscle protein degradation and/or maintaining lean body mass in a subject wherein combination is as described herein elsewhere.

In the third and fourth aspects of the present invention, the subject is preferably a human, more preferably an elderly human.

All embodiments of the combinations described herein in connection with the first aspect of the invention (see section B) are equally applicable to the methods and combinations for use according to the fourth and fifth aspects of the present invention.

Muscle protein balance is a key aspect of overall health, and muscle health in particular. Generally, the metabolic pathways that affect muscle protein balance are tightly regulated. However, such pathways can become dysregulated due to certain disease conditions, prolonged periods of physical inactivity, and the aging process. One particularly negative aspect resulting from such pathway dysregulation is the loss of lean body mass.

Preserving lean body mass is important for maintaining the strength necessary to carry out activities of daily living, particularly with respect to the elderly population. Additionally, severe loss of lean body mass can result in an increased risk of infection and marked delays in wound healing (Pereira et al., The International Journal of Biochemistry & Cell Biology, Vol. 37: 1948-1961 (2005)). Conventionally, loss of lean body mass has been treated with nutritional interventions designed to enhance muscle protein synthesis. These nutritional interventions may be embodied as oral nutrition supplements that are chronically administered to a subject exhibiting loss of lean body mass. However, such interventions designed for stimulating muscle protein synthesis may be inadequate for effectively preserving lean body mass since the rate of muscle protein degradation is not addressed by such nutritional interventions.

Numerous studies have been performed to examine the effects of nutritional interventions on stimulating muscle protein synthesis. These studies have generally demonstrated that high levels of protein {e.g., greater than 12% of total caloric intake) or branched chain amino acids are effective for stimulating muscle protein synthesis, particularly in elderly subjects. On the other hand, the anabolic response of skeletal muscle of compromised subjects (e.g., elderly subjects, diseased subjects) is considerably attenuated compared to non-compromised subjects {e.g., young subjects, non-diseased subjects) (Guillet et al, FASEB J, Vol. 18: 1586-1587 (2004)). The observed attenuation of the anabolic response may be due to increased muscle protein degradation via the ubiquitin-proteasome pathway. For example, ubiquitin-proteasome pathway dependent protein degradation has been associated with the loss of lean body mass in diseased states such as chronic obstructive pulmonary disease (COPD) (Debigare et al., Proc. Am. Thorac. Soc, Vol. 7: 84-90 (2010)) and chronic renal failure (Debigare et al, Am. J. Physiol. Renal Physiol., Vol. 285: FI-8 (2003)). Thus, effective interventions for preserving lean body mass need to address each component of the muscle protein balance equation.

Accordingly, methods and nutritional combinations for attenuating muscle protein degradation, preserving lean body mass, or both are provided herein. The exemplary methods and pharmaceutical or nutritional combinations described herein may be effective for preserving lean body mass by attenuating muscle protein degradation.

In some exemplary embodiments, the subject in need thereof is suffering from sarcopenia. Sarcopenia is the term typically used to describe the degenerative loss of skeletal muscle mass and strength associated with aging. It is a condition that is typically not caused by or associated with other pathological conditions and therefore can affect otherwise healthy individuals. In certain exemplary embodiments, methods of preventing or treating sarcopenia involve administering a combination as disclosed herein to an elderly subject who is otherwise healthy, particularly an elderly subject who does not suffer from any other muscle-wasting disease or condition.

In certain exemplary embodiments, the subject in need thereof is suffering from a muscle wasting disease. The term “muscle wasting disease” is used herein to mean any disease wherein the patient suffers from muscle atrophy as a cause or consequence of the pathology. “Cachexia” is a term used to describe the muscle wasting that occurs in individuals suffering from a number of different diseases. Thus, in certain embodiments, provided herein are methods of preventing or treating cachexia, optionally cachexia associated with one or more of the following diseases: cancer, human immunodeficiency virus/acquired immune deficiency syndrome (HIV/AIDS), autoimmune disease including multiple sclerosis, chronic obstructive pulmonary disease (COPD), end stage renal disease (ESRD), tuberculosis, congestive heart failure and familial amyloid polyneuropathy.

In certain exemplary embodiments, the combination is administered to a human subject such that the amount of HMB consumed by the subject ranges from about 10 mg/kg/day to about 40 mg/kg/day, including from about 20 mg/kg/day to about 30 mg/kg/day, and including from about 23 mg/kg/day to about 27 mg/kg/day. Thus, the amount of HMB consumed by the subject is significantly less than the minimum recommended dose for attenuating muscle protein degradation or preserving lean body mass (50 mg/kg/day). In certain exemplary embodiments, the combination is provided in one or multiple (e.g., two, three, four) servings per day to achieve the desired amount of HMB. In certain exemplary embodiments, the combination is provided in two servings per day to achieve the desired amount of HMB.

In certain exemplary embodiments, the combination is administered to a human subject such that the amount of THC consumed by the subject ranges from about 5 mg/kg/day to about 40 mg/kg/day, including from about 10 mg/kg/day to about 25 mg/kg/day, and including from about 15 mg/kg/day to about 20 mg/kg/day. In certain exemplary embodiments, the combination is provided in one or multiple (e.g., two, three, four) servings per day to achieve the desired amount of THC. In certain exemplary embodiments, the combination is provided in two servings per day to achieve the desired amount of THC.

In certain exemplary embodiments, the combination is administered to a human subject such that the amount of HMB consumed by the subject ranges from about 20 mg/kg/day to about 30 mg/kg/day, and the amount of THC consumed by the subject ranges from about 10 mg/kg/day to about 25 mg/kg/day.

In certain exemplary embodiments, the combination is administered to a human subject such that the amount of HMB consumed by the subject ranges from about 23 mg/kg/day to about 37 mg/kg/day, and the amount of THC consumed by the subject ranges from about 15 mg/kg/day to about 20 mg/kg/day.

As will be appreciated, providing the HMB and THC in separate compositions allows the HMB and THC to be administered sequentially. It also allows the HMB and THC to be administered simultaneously, for example where the user mixes the separate compositions prior to administration. Providing the HMB and THC in a single composition allows for the simultaneous administration of HMB and THC.

In certain exemplary embodiments of the methods described herein, the combination is administered to the subject in need thereof for a period of at least three weeks. For example, the combination or combination, in certain exemplary embodiments, may be administered to the subject in need thereof for a period of three to eight weeks, three weeks to six months, three weeks to nine months, or three weeks to one year or even longer. In certain other exemplary embodiments, the effective amount of the or combination is administered to the subject in need thereof for a period of time of up to three weeks, such as one day, two days, three days, four days, five days, six days, one week, ten days, two weeks, or three weeks.

In accordance with certain exemplary embodiments of the methods disclosed herein, the effective amount of the combination can be administered to the subject in need thereof one or more times per day for a period of up to three weeks, or for a period of at least three weeks, to achieve the desired effect. For example, in certain exemplary embodiments, the effective amount of the combination can be administered to the subject in need thereof every day for at least three weeks, every day for at least four weeks, every day for at least eight weeks, every day for at least six months, or every day for a year or more. As another example, the effective amount of the combination can be administered to the subject in need thereof twice a day for at least three weeks, twice a day for at least four weeks, twice a day for at least eight weeks, twice a day for at least six months, or twice a day for a year or more. Within the context of providing a serving to the subject in need thereof, every day is intended to reflect a subject who has been instructed to be administered the exemplary combination as described herein every day and who actually is administered the combination for at least 70% (and in certain other exemplary embodiments at least 90%) of the days during the period of administration.

In certain exemplary embodiments, the combination is acutely administered to the subject in need thereof. The phrases “acutely administered,” “acute administration,” or “acutely administering,” as used herein, refer to administering an effective amount of the exemplary nutritional combination to the subject in need thereof on a non-regular basis. Acute administration may be a single serving, or multiple servings, administered over a relatively short time period, such as up to three weeks, including one day, two days, three days, five days, one week, ten days, two weeks, or three weeks.

In certain exemplary embodiments, the combination is chronically administered to the subject in need thereof. “Chronically administering,” as used herein, refers to regular administration which is provided indefinitely or to regular administration for a significant period of time. For example, in certain exemplary embodiments, chronic administration can include regular administration for at least three weeks, regular administration for at least one month, regular administration for at least 6 weeks, regular administration for at least two months, regular administration for at least 3 months, regular administration for at least 4 months, regular administration for at least 5 months, regular administration for at least 6 months, or regular administration for at least 9 months. In other exemplary embodiments, the chronic administration refers to regular administration for at least 1 year, regular administration for at least 1.5 years, regular administration for at least 2 years, or regular administration for more than 2 years. “Regular administration,” as used herein, refers to administration according to a schedule whereby the subject in need thereof will receive the exemplary nutritional combination at regular intervals.

As used herein, “regular intervals” refers to administration in a repeating, periodic fashion where the time between administrations is approximately (or intended to be approximately) the same. In various exemplary embodiments, administration at regular intervals includes daily administration or weekly administration. In other exemplary embodiments, administration at regular intervals includes administration 1-2 times per week, administration 1-3 times per week, administration 2-3 times per week, administration 1-4 times per week, administration 1-5 times per week, administration 2-5 times per week, administration 3-5 times per week, administration 1-6 times per week, administration 1-7 times per week, administration 2-6 times per week, administration 2-7 times per week, administration 1-2 times per day, administration 1-3 times per day, administration 1-4 times per day, administration 2-3 times per day, administration 2-4 times per day, administration 3-4 times per day, administration 2-5 times per day, administration 3-5 times per day, or administration 4-5 times per day.

The lean body mass of a subject may be determined by any suitable method for determining a subject's lean body mass. For example, lean body mass may be determined by methods including, but not limited to, bioelectrical impedance analysis (BIA), air displacement plethysmography (ADP), hydrodensitometry, dual energy x-ray absorptiometry (DEXA), densitometry, magnetic resonance imaging (MRI), computed tomography (CT), and combinations thereof.

In certain exemplary embodiments, preserving lean body mass means the lean body mass of a subject decreases by no more than a certain percentage, preferably no more than 10%, more preferably no more than 5%. In certain exemplary embodiments, preserving lean body mass means the lean body mass of the subject is maintained within a certain percentage of the subject's lean body mass prior to consumption of the nutritional combination, preferably within 10%, more preferably within 5%. In certain exemplary embodiments, a pre-administration measurement of lean body mass takes place within 1 week prior to initiation of the administration of the nutritional combinations disclosed herein. In certain such embodiments, the measurement takes place within 1-7 days prior to initiation of the administration.

In other exemplary embodiments, preserving lean body mass means an increase in the lean body mass of the subject as compared to the lean body mass maintained by the subject without administration of the nutritional combination, preferably at least 5%, more preferably at least 10%.

Notably, it is intended that preserving lean body mass by consumption of one or more of the exemplary nutritional combinations described herein may be illustrated via an animal study, wherein the animals consume an equivalent amount of the same nutritional combination for a certain time period.

It should be noted that the methods for attenuating muscle protein degradation and the methods for maintaining lean body mass provided herein are freely combinable. Indeed, the methods may be effective for preserving lean body mass by attenuating muscle protein degradation.

The methods described herein may involve administering a combination as disclosed herein elsewhere to a subject as part of a regular meal or to replace a regular meal. For example, the combination may serve as a supplemental nutritional source or the sole source of nutrition for the subject. Wherein the combination is administered as the sole source of nutrition, this may be the sole source of nutrition for a single meal or may be the sole source of nutrition replacing the subject's normal nutritional intake over a defined period, for example, a period of at least 8 hours, a period of at least 24 hours, a period of at least 7 days.

For embodiments wherein the combination is administered as part of a regular meal, the combination may be administered, for example consumed, prior to or during consumption of the meal. In embodiments wherein the combination is administered to replace a regular meal or is intended to serve as the sole source of nutrition for the subject, the combination may be formulated as a nutritional product intended to provide a certain amount of energy per serving. For example, the nutritional combination may be formulated so as to provide up to 500 kcal of energy per serving, including from 20 kcal to 500 kcal, from 75 kcal to 500 kcal, from 150 kcal to 500 kcal, from 200 kcal to 500 kcal, from 300 kcal to 500 kcal, or from 400 kcal to 500 kcal per serving.

The invention will now be understood with reference to the following non-limiting examples.

### EXAMPLES

### Example 1

A study was carried out to investigate the effect of a composition comprising HMB and white curcumin (THC) in a well-established animal model for human muscle loss (β-Hydroxy-β-methylbutyrate (HMB) normalizes dexamethasone-induced autophagy-lysosomal pathway in skeletal muscle. Giron M D, Vilchez J D, Shreeram S, Salto R, Manzano M, Cabrera E, Campos N, Edens N K, Rueda R, Lopez-Pedrosa J M. PLoS One. 2015 Feb. 6; 10(2):e0117520. doi: 10.1371/journal.pone.0117520. eCollection 2015).

Experimental design—Male Sprague-Dawley rats (8-10 weeks old) (n=42) having a body weight of 270-310 g were randomly assigned to seven experimental groups (six rats per group). Groups 1-6 were administered 0.1 mg/kg/day dexamethasone (a synthetic glucocorticoid) by intraperitoneal injection for 21 days to induce muscle loss. The dexamethasone was administered at 2 mL/kg of 1.75 mg dexamethasone in 35 mL saline. Group 7 (a sham control) was administered saline without the dexamethasone.

Group 1 (a pathological control) was administered the 0.1 mg/kg/day dexamethasone alone. For groups 2-6 the dexamethasone was co-administered with HMB, white curcumin or both in the following dosages:
Group 2: 160 mg/kg/day HMB
Group 3: 320 mg/kg/day HMB
Group 4: 100 mg/kg/day white curcumin
Group 5: 200 mg/kg/day white curcumin
Group 6: 160 mg/kg/day HMB+100 mg/kg/day white curcumin

The rats were fed AIN 93-M diet.

The body weight of each rat was measured daily. On day 21 the soleus and rectus femoris muscles from each rat were collected and weighed and the lean mass of each rat was recorded. The fore limb and hind limb grip strengths were also measured.

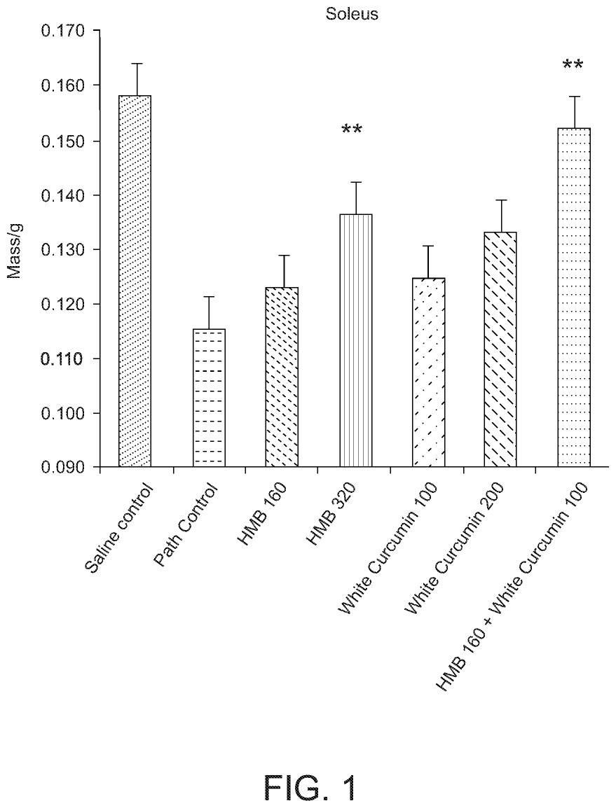

Results—as shown in FIG. 1, only the co-administration of 100 mg/kg/day white curcumin and 160 mg/kg/day HMB, or 320 mg/kg/day HMB, exhibited a significant difference relative to the pathological control. 200 mg/kg/day white curcumin administered alone did not have a significant effect relative to the pathological control.

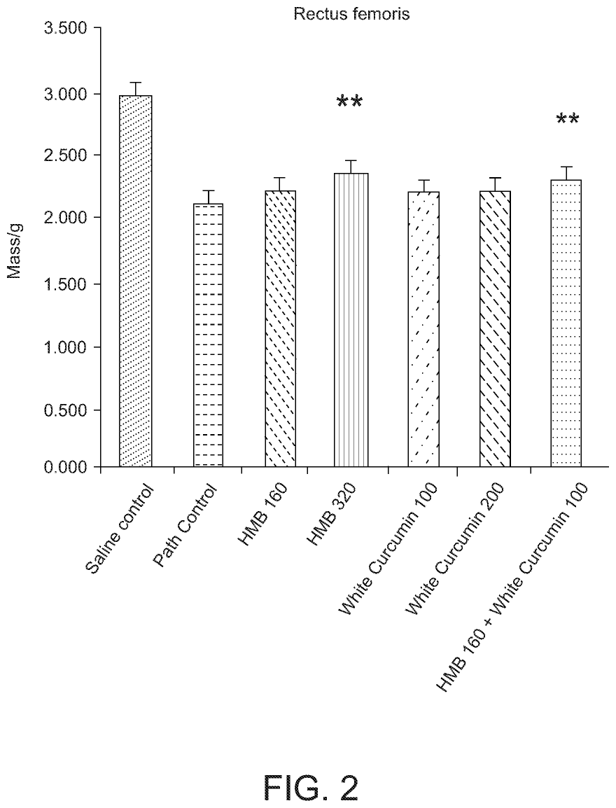

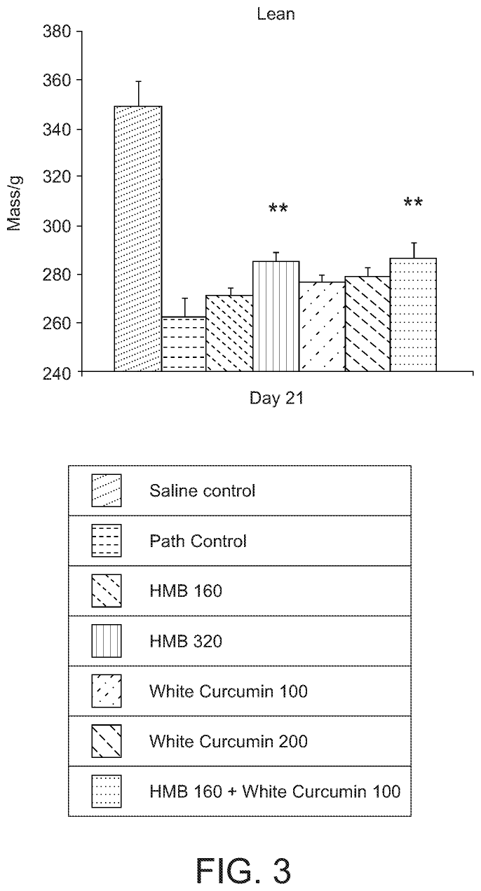

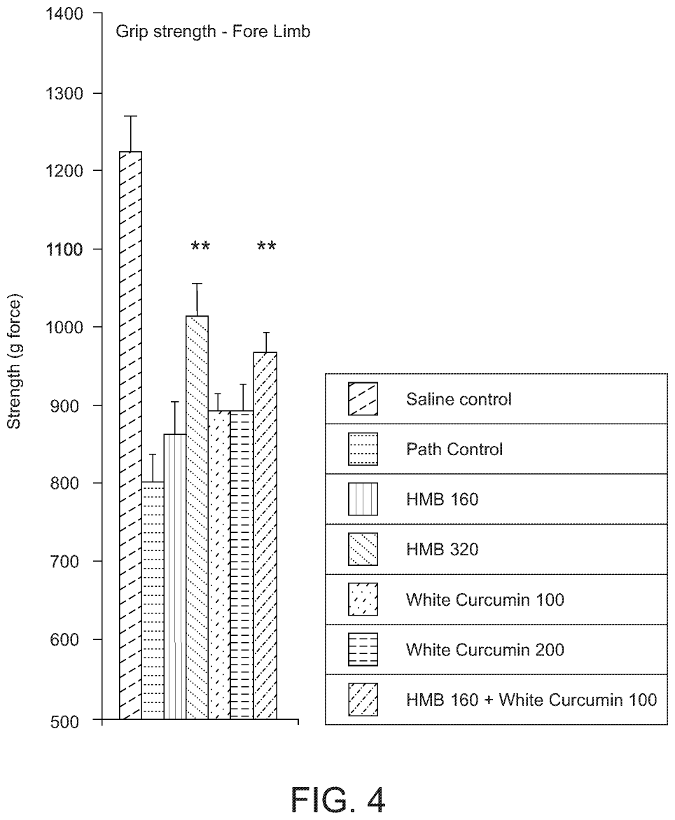

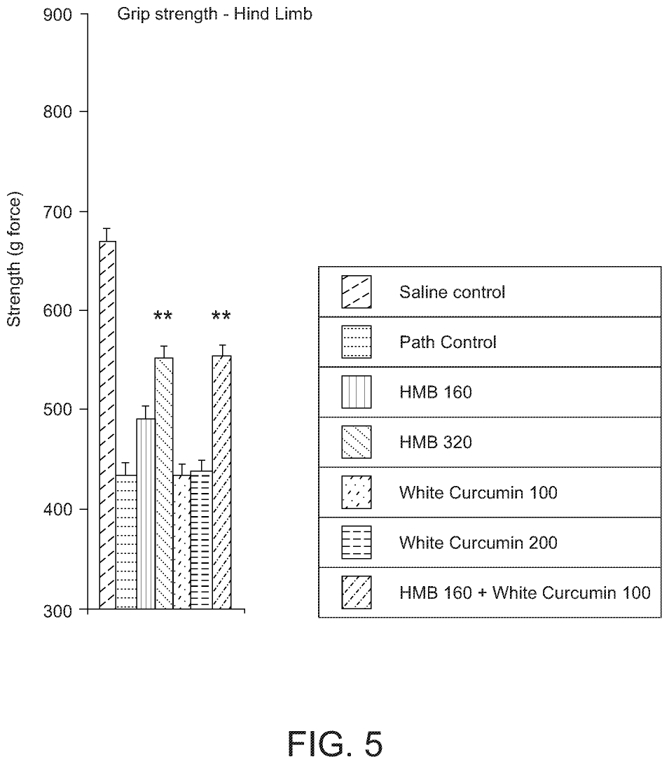

The data for rectus femoris muscle mass (FIG. 2), lean body mass (FIG. 3), fore limb grip strength (FIG. 4), and hind limb grip strength (FIG. 5) illustrate a similar trend.

In the figures, a double asterisk ** identifies a statistically significant difference relative to the pathological control (P<0.05), as calculated by t-test. The error bars indicate standard error of the mean (SEM).

### Example 2

Example 2 illustrates a nutritional powder in accordance with the present disclosure, the ingredients of which are listed in the table below. The product was prepared by a spray-drying method. All ingredients are listed as kg per 1000 kg batch of product, unless otherwise specified. A comparison is given with a known HMB-containing nutritional powder.

| | | Comparative |
|---|---|---|
| Ingredient | Example 2 | Example 2 |
| Maltodextrin | 436.7 | 436.7 |
| Sucrose | 145.5 | 145.5 |
| Calcium Caseinate | 129.1 | 129.1 |
| Isolated Soy Protein | 57.7 | 57.7 |
| FOS Powder | 33.6 | 33.6 |
| HO sunflower oil | 59.9 | 59.9 |
| Calcium HMB | 15.8 | 31.6 |
| THC | 9.9 | 0 |
| Canola Oil | 55.1 | 55.1 |
| Soy Oil | 26.7 | 26.7 |
| Potassium Citrate | 10.3 | 10.3 |
| Sodium Citrate | 5.8 | 5.8 |
| Potassium Chloride | 5.2 | 5.2 |
| Magnesium Chloride | 4.7 | 4.7 |
| Potassium hydroxide | 3.1 | 3.1 |
| Sodium phosphate dibasic dihydrate | 3.0 | 3.0 |
| Sodium chloride | 2.5 | 2.5 |
| Choline Chloride | 1.8 | 1.8 |
| Vanilla Flavor | 1.8 | 1.8 |
| Sodium phosphate monobasic monohydrate | 1.6 | 1.6 |
| Potassium phosphate dibasic trihydrate | 1.1 | 1.1 |
| Flavor | 1.0 | 1.0 |
| Vitamin premix | 1.0 | 1.0 |
| Ascorbyl palmitate | 0.243 | 0.243 |
| Ascorbic acid | 0.240 | 0.240 |
| Antioxidant | 0.116 | 0.116 |
| Ferrous sulfate | 0.010 | 0.010 |
| Vitamin premix | 0.065 | 0.065 |
| Zinc sulfate monohydrate | 0.057 | 0.057 |
| Manganese sulfate | 0.045 | 0.045 |
| Mineral mix copper sulfate | 0.035 | 0.035 |
| Beta carotene | 0.005 | 0.005 |
| Chromium chloride | 0.001 | 0.001 |
| Sodium molybdate | 0.0012 | 0.0012 |
| Potassium iodide | 0.001 | 0.001 |
| Sodium selenite | 0.0004 | 0.0004 |
| Citric acid | AN | AN |
| Potassium hydroxide | AN | AN |
| Magnesium sulfate dry | AN | AN |
| Ultra micronized tricalcium phosphate | AN | AN |
| Ascorbic acid | AN | AN |

### Example 3

Example 3 illustrates a nutritional liquid in accordance with the present disclosure, the ingredients of which are listed in the table below. The product was prepared by a spray-drying method. All ingredients are listed as kg per 1000 kg batch of product, unless otherwise specified. A comparison is given with a known HMB-containing nutritional liquid.

| | | Comparative |
|---|---|---|
| Ingredient | Example 3 | Example 3 |
| Water | Q.S. | Q.S. |
| Sucrose | 89.3 | 89.3 |
| Maltodextrin | 29.7 | 29.7 |
| Sodium Caseinate | 25.9 | 25.9 |
| Milk Protein Concentrate | 19.1 | 19.1 |
| Soy Protein Isolate | 11.9 | 11.9 |
| Potassium Citrate | 7.9 | 7.9 |
| Soy Oil | 11.1 | 11.1 |
| Calcium HMB | 3.4 | 6.7 |
| THC | 2.1 | 0 |
| Canola Oil | 10.2 | 10.2 |
| Corn Oil | 9.3 | 9.3 |
| Whey Protein Concentrate | 3.5 | 3.5 |
| Magnesium Phosphate Dibasic | 3.1 | 3.1 |
| Flavoring Agent | 2.0 | 2.0 |
| Stabilizer | 2.0 | 2.0 |
| Soy Lecithin | 1.5 | 1.5 |
| Sodium Phosphate Dibasic Dihydrate | 1.3 | 1.3 |
| Potassium Phosphate Dibasic | 0.985 | 0.985 |
| Potassium Chloride | 0.729 | 0.729 |
| Choline Chloride | 0.480 | 0.480 |
| Ascorbic Acid | 0.469 | 0.469 |
| Calcium Carbonate | 0.451 | 0.451 |
| Caramel Flavor | 0.450 | 0.450 |
| Dairy Creamer | 0.450 | 0.450 |
| UTM/TM Premix | 0.367 | 0.367 |
| 45% Potassium Hydroxide | 0.323 | 0.323 |
| Carrageenan | 0.200 | 0.200 |
| Water Soluble Vitamin Premix | 0.185 | 0.185 |
| Vitamin DEK Premix | 0.067 | 0.067 |
| Sodium Chloride | 0.060 | 0.060 |
| Gellan Gum | 0.050 | 0.050 |
| Vitamin A Palmitate | 0.0082 | 0.0082 |
| Corn oil carrier | Q.S. | Q.S. |
| Vitamin D₃ | 399 mg | 399 mg |
| Potassium Iodide | 194 mg | 194 mg |

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

1. A pharmaceutical or nutritional combination comprising beta-hydroxy betamethylbutyrate (HMB) and tetrahydrocurcumin (THC).

2. The combination according to claim 1, wherein the combination has a weight ratio of HMB to THC of from 1:1 to 3:1.

3. The combination according to claim 1, wherein the combination comprises from 0.5 to 1.5 wt % THC by dry weight of the combination.

4. The combination according to claim 1, wherein the combination comprises from 1 to 2 wt % HMB by dry weight of the combination.

5. The combination according to claim 1, wherein the HMB and THC are provided in separate compositions.

6. The combination according to claim 1, wherein the HMB and THC are provided in a single composition.

7. A method of manufacturing the combination according to claim 6, comprising mixing a source of HMB with a source of THC.

8. A method of treatment of a subject by therapy, the method comprising administering to the subject a combination according to claim 1.

9. (canceled)

10. A method of attenuating muscle protein degradation and/or maintaining lean body mass in a subject in need thereof, the method comprising administering to the subject a combination according to claim 1.

11. (canceled)

12. The method according to claim 8, wherein the subject in need thereof is an elderly human.

13. The method according to claim 8, wherein the subject in need thereof is suffering from sarcopenia or a muscle wasting disease.

14. The method according to claim 8, wherein the combination is administered to a human subject such that the amount of THC consumed by the subject ranges from about 10 mg/kg/day to about 25 mg/kg/day.

15. The method according to claim 8, wherein the combination is administered to a human subject such that the amount of HMB consumed by the subject ranges from about 20 mg/kg/day to about 30 mg/kg/day.

16. The method according to claim 10, wherein the subject in need thereof is an elderly human.

17. The method according to claim 10, wherein the subject in need thereof is suffering from sarcopenia or a muscle wasting disease.

18. The method according to claim 10, wherein the combination is administered to a human subject such that the amount of THC consumed by the subject ranges from about 10 mg/kg/day to about 25 mg/kg/day.

19. The method according to claim 10, wherein the combination is administered to a human subject such that the amount of HMB consumed by the subject ranges from about 20 mg/kg/day to about 30 mg/kg/day.

20. The combination according to claim 2, wherein the combination comprises from 0.5 to 1.5 wt % THC by dry weight of the combination.

21. The combination according to claim 2, wherein the combination comprises from 1 to 2 wt % HMB by dry weight of the combination.
